(19) United States
(12) Patent Application Publication
(10) Pub. No.: US 2024/0363017 A1
(43) Pub. Date: Oct. 31, 2024

(54) INTERACTIVE TOOL FOR TREATMENT OF CHILDHOOD APRAXIA OF SPEECH

(71) Applicant: Apraxia Kids, Pittsburgh, PA (US)

(72) Inventors: Amanda Stein, Pittsburgh, PA (US); Lou LaVecchia, Hebron, CT (US); Michelle Leigh, Jacksonville Beach, FL (US); Paula Hill, Dallas, TX (US); Jessica Carrizo, Davis, CA (US); Earnie Sotirokos, Pittsburgh, PA (US); Audrey Swanderski, Pittsburgh, PA (US); Laura Moorer, Denton, TX (US); Melissa Gillmer, Orange Park, FL (US)

(73) Assignee: Apraxia Kids, Pittsburgh, PA (US)

(21) Appl. No.: 18/646,311

(22) Filed: Apr. 25, 2024

Related U.S. Application Data
(60) Provisional application No. 63/462,017, filed on Apr. 26, 2023.

(51) Int. Cl. G09B 5/02; A61B 5/00

(52) U.S. Cl. CPC G09B 5/02; A61B 5/4842

(57) ABSTRACT

An example method includes a method, including: accessing a graphical user interface (GUI) comprising an interactive tool for assisting a user seeking assistance or support for a child with a childhood neurological motor planning disorder; providing, via the GUI, a first input indicative of a first progress state of the child; receiving, via the GUI, one or more recommendations for speech services for the child; applying the one or more recommendations to the child; subsequently providing, via the GUI, a second progress state of the child; and receiving, via the GUI, one or more updated recommendations for the child.

### CROSS-REFERENCE TO RELATED APPLICATIONS

This application claims the benefit of U.S. provisional patent application Ser. No. 63/462,017, having the title “INTERACTIVE GRAPHICAL USER INTERFACE (GUI) TO FACILITATE GUIDED NAVIGATION AND DIRECT-ACCESS TO DATABASE SUBSETS” and filed on 26 Apr. 2023, the contents of which are incorporated by reference in their entirety herein.

### COPYRIGHT NOTICE

A portion of the disclosure of this patent document contains material to which a claim for copyright is made. The copyright owner has no objection to the facsimile reproduction by anyone of the patent document or the patent disclosure, as it appears in the Patent and Trademark Office patent file or records, but reserves all other copyright rights whatsoever.

### BACKGROUND

A vast amount of information related to assisting in educational access and treatment plans for childhood apraxia of speech may be stored as electronic content such as documents in one or more databases. Moreover, users of conventional search interfaces and applications may need to periodically access particular content as they perform tasks. For example, a user working to identify requisite policy documents, regulations, guidance documents, or similar data might need to access documents containing eligibility policies, standards, definitions, rules, etc. Navigation to the appropriate content, however, can be a time consuming and error prone task, especially when there are a substantial number of documents or content items that are potentially relevant. For example, documents might be stored in a hierarchical tree or file structure that requires a user to make many selections and have familiarity with the file system before eventually reaching a needed piece of information. These issues are compounded where more than one source or database needs to be accessed to formulate a solution to a given task, with the relevant content changing based on the context in which the data is accessed.

Conventionally, software may guide users, for example by providing a keyword or faceted searching functionality. Nonetheless, having highly educated users with advanced domain knowledge remains a requirement for using such tools to navigate complex data assets, for example with the ability to pick relevant keywords or phrases, understand relevancy of search results, or even applicability if the documents or content is frequently updated, modified, or variably applicable, for example in different jurisdictions. Examples of such domain knowledge typically include things such as medicine and the sciences. However, equally complex data assets and specialized knowledge domains must be utilized to determine, for example, eligibility for certain programs or education or health benefits, and/or individual educational plans (IEPs). As such, related tasks retain a requirement to have users with advanced expertise that they must apply as they navigate the data assets.

### BRIEF SUMMARY

In summary, an embodiment provides a method, comprising: accessing a graphical user interface (GUI) comprising an interactive tool for assisting a patient with a childhood neurological motor planning disorder; providing, via the GUI, a first input indicative of a first progress state of the patient; receiving, via the GUI, one or more recommendations for speech services for the patient; applying the one or more recommendations to the patient; subsequently providing, via the GUI, a second progress state of the patient; and receiving, via the GUI, one or more updated recommendations for the patient.

In an embodiment, the childhood neurological motor planning disorder is childhood apraxia of speech.

In an embodiment, the one or more recommendations for speech services comprise at home speech practice.

In an embodiment, the method comprises providing, via the GUI, second input to one or more nodes representative of individual educational plan development stage.

In an embodiment, the method comprises obtaining, via the GUI, content associated with a respective one of the one or more nodes associated with the second input.

In an embodiment, the one or more nodes are logically associated along a guidance path.

In an embodiment, the guidance path comprises a path for forming or updating an individual educational plan comprising the speech service.

In an embodiment, the method comprises receiving automated animation of displayed content along the guidance path in response to user input.

In an embodiment, the method comprises obtaining a saved position in the guidance path for a user.

In an embodiment, the method comprises obtaining a customization of the guidance path based on one or more user characteristics of a user.

A further embodiment provides a computer program product comprising a non-transitory computer readable medium storing processor-executable code configured to execute one or more methods, or parts thereof, as shown and described.

A yet further embodiment provides a system of one or more devices configured to perform one or more methods, or parts thereof, as shown and described.

The foregoing is a summary and is not intended to be in any way limiting. For a better understanding of the example embodiments, reference can be made to the detailed description and the drawings. The scope of the invention is defined by the claims.

### BRIEF DESCRIPTION OF THE SEVERAL VIEWS OF THE DRAWINGS

FIG. 1 illustrates an example of part of a data model according to an embodiment.

FIG. 2(A-B) illustrates a part of an interactive GUI according to an embodiment.

FIG. 3 illustrates an example method according to an embodiment.

FIG. 4 illustrates an example method according to an embodiment.

FIG. 5 illustrates an example system according to an embodiment.

### DETAILED DESCRIPTION

It will be readily understood that the components of the embodiments, as generally described and illustrated in the figures herein, may be arranged and designed in a wide variety of ways in addition to the examples described herein. The detailed description uses examples, represented in the figures, but these examples are not intended to limit the scope of the claims.

Reference throughout this specification to “embodiment(s)” (or the like) means that a particular described feature or characteristic is included in that example. The particular feature or characteristic may or may not be claimed. The particular feature may or may not be relevant to other embodiments. For the purpose of this detailed description, each example might be separable from or combined with another example.

Therefore, the described features or characteristics of the examples generally may be combined in any suitable manner, although this is not required. In the detailed description, numerous specific details are provided to give a thorough understanding of example embodiments. One skilled in the relevant art will recognize, however, that the claims can be practiced without one or more of the specific details found in the detailed description, or the claims can be practiced with other methods, components, etc. In other instances, well-known details are not shown or described to avoid obfuscation.

It would be desirable to provide systems, methods, and products to facilitate content navigation in complex domains while allowing accurate, efficient, and structured paths for effective creation, review, and/or monitoring of associated content navigation paths for task completion, such as formulating, implementing, or updating an individual education plan (IEP), completing an insurance eligibility assessment, etc. As used herein, the term “content” may refer to, for example, any digital content, such as a text document, electronic file, image, video, query, spreadsheet, etc., contained for example in a database storing electronic or digital data content and associated metadata.

An embodiment provides a method for employing a data model formed from a complex content set, used to configure an interactive graphical user interface (GUI) tool that controls and guides content navigation in a task-specific, contextualized manner. For example, a user might provide an indication via the interactive GUI, such as a role or a context that indicates that the user is preparing for a specific task, such as an IEP meeting with educational institution representatives in which an IEP will be formed, evaluated and/or updated to track progress of a patient receiving speech services as part of a therapeutic and educational program.

In an embodiment, a computer system implementing the interactive GUI, such as computer system hosting an interactive web application in a browser, a mobile phone hosting a native mobile application, etc., may access or reference an associated data model that defines a relationship between elements of content, such as one or more source electronic data records, and interface elements. According to some embodiments, a portion of content, such as an electronic data record, may be associated with a corresponding data model that defines a relationship between the portion of the content, such as a text string, image, video, etc., and an element in the interactive GUI. Based on the context, such as user role, a user's response to an initial question, a user's geographic location (such as gathered from a device subsystem or service like global positioning satellite (GPS) data, session data, etc.,) an embodiment associates the correct subset of the electronic content item(s) with the respective element(s) in the interactive GUI to provide displayable data object(s) with which the user may interact.

By way of non-limiting example, an embodiment may select for display an initial portion of the electronic content associated with a respect element of the interactive GUI that is chosen for display based on the user indication(s). In an embodiment, a user may navigate along one or more paths to display associated data objects, as defined by the respective data model, i.e., without a need to perform subsequent searches or refinements. That is, the interactive GUI loads the one or more possible navigation paths through the content for the user, guiding the user to specific parts among a larger source corpus. In this way, an embodiment facilitates efficient user interaction with a larger pool of content without requiring the user to know or understand relevant search terms, search facets, file structures, or available entry points into the data.

An embodiment provides a method for employing a data model formed from a complex content set, used to configure an interactive graphical user interface (GUI) that controls and guides content navigation in a task-specific, contextualized manner. For example, a user might provide an indication of a role or context, such as a user preparing for a specific task like an IEP meeting with educational institution representatives. In an embodiment, a computer system implementing the interactive GUI, such as an interactive web application hosted in a browser, a native mobile application, or a single page application, may access or reference an associated data model that defines a relationship between elements of one or more source content items and interactive elements of the one or more paths defined by the data model. An embodiment associates the correct subset of the electronic content with the respective element in the interactive GUI for initial display and restricts the user's movement through the content to the one or more paths defined by the data model.

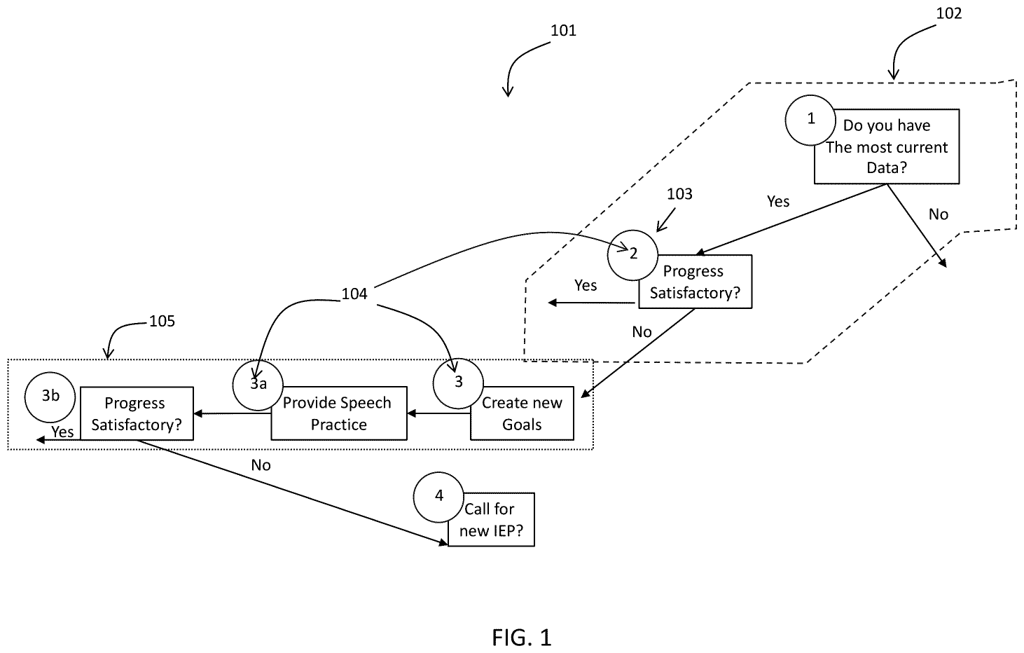

Referring to FIG. 1, an example portion of a data model 101 is illustrated. In the example of FIG. 1, the portion of the data model 101 is from a data model suitable for use in planning and implementing an IEP, although it is noted here that other data models, e.g., for insurance eligibility, etc., may be utilized. The illustrated example portion of the data model 101 of FIG. 1 is part of an example data model provided for descriptive purposes as part of this disclosure.

As illustrated, the portion of the data model 101 defines distinct navigation pathways, illustrated by way of example as bounded by boxes 102 and 105. These navigation pathways are topically or logically associated with content selected from a larger data source, such as legislation, regulations, policies, as well as guides and interpretations. The pathways, e.g., 102, 105, are configured to guide the user along a logical selection of curated content suited for a particular task or subtask, for example prepared by an expert with domain knowledge. It is noted that the larger data source may comprise one or more sources of data and may include supplemental content, for example supplied by a domain expert, expert system, automated summary service, or the like.

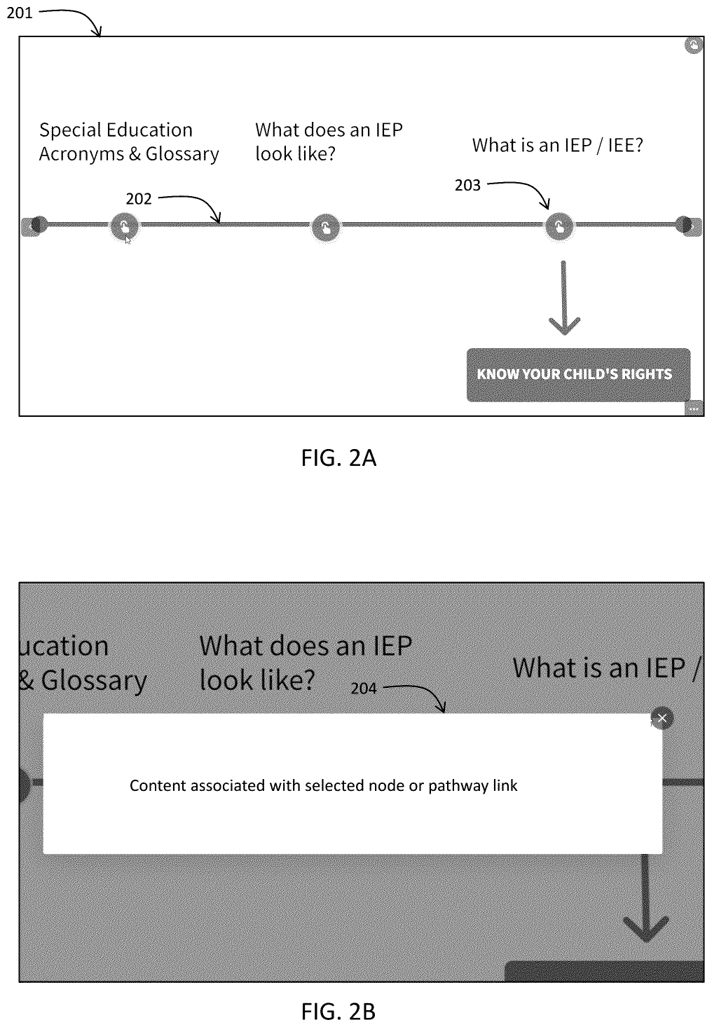

The example of FIG. 1 illustrates that the portion of the data model 101 and respective pathways, e.g., pathway 102, are associated with nodes, such as indicated at 103, in turn related to displayable elements or data objects of an interactive GUI (described in more detail in the example of FIG. 2A-B). As indicated at 104, the nodes may be associated with metadata that facilitates quick retrieval of content associated with a given node, e.g., node 103. As such, user interaction with a respective displayable element or data object presented in the interactive GUI allows an embodiment to retrieve the associated content, e.g., by reference to metadata 104 such as node ID, hierarchy level (e.g., 3), subcategory (e.g., a, b, c, etc.) and the like. As may be appreciated by those having skill in the art, use of metadata in association with the model allows the content selected to be indirectly associated with a node, e.g., referenced by table lookup with a corresponding column and/or row of source content in a table, which permits varying the content referenced without modifying the model characteristics, e.g., the pathways, some of which are indicated at 102 and 105, and the related elements of the interactive GUI.

Continuing with the example of FIG. 1, the content associated with a given node such as 103, and reference by or called using metadata 104, may be a subset of a larger data source. In one example, the subset of content is summarized content, for example a textual summary of source legislation, regulation, policy, interpretation or guidance. In some embodiments, the summary content may be prepared manually. In an embodiment, the summary is prepared automatically, for example via use of an application programming interface (API) call to an external source, such as a transformer based artificial intelligence (AI) model, such as a generative pretrained transformer (GPT) model, which summarizes data given a data prompt, e.g., a snippet of source data text as input, and returns the data summary as output. As will be appreciated, a combination of the foregoing techniques may be utilized.

Review of the examples illustrated in FIG. 2(A-B) indicates that a portion of the data model 101 includes pathways, some of which are highlighted at 102 and 105, that are associated with visually highlighted pathways and display objects related thereto. In data model 101, the pathways define a relationship between the nodes, e.g., 103, elements of the interactive GUI, e.g., 203, and linkages there between, e.g., 202. This permits an embodiment to guide the user through the content in a curated or designed manner, navigating between nodes along the pathway, removing a need to perform searches such as via use of keywords, facets, etc. In some examples, interaction with a node moves the user to another part of the pathway, displayed in a separate page or view, or to another pathway. Interactive GUI 201 further visually indicates relationships between interactive data objects, e.g., 203, such that the user is provided with a visual indication of the underlying data relationships as defined by model 101. In some embodiments, the source data and/or other data may be made available to the user, e.g., via interactive GUI 201, for example by using an “additional sources” node that is always accessible, etc.

As shown in FIG. 2A, an embodiment may display interactive GUI 201 with which a user may interface to navigate along the pathways defined by the model, which may be graphically represented in the interactive GUI, e.g., as indicated at 202. Similarly, the nodes of the model, for example node 103, are graphically represented as interface elements in interactive GUI 201, e.g., as indicated at 203. This visually presents the modeled pathway to the user, assisting the user in making selections from among the various selectable elements. As may be appreciated, contextual data, such as stored session data, user profile data, role or credentials of a user, group membership, geographic location (as determined via a suitable method such as GPS, IP address, proximity sensing via wireless sensors or radios, etc.) or similar may be used to position the user dynamically in the pathway and focus one or more nodes for the user's attention, i.e., dynamically update the displayed data objects for the user. In a similar fashion, the contextual data may also be utilized to select or modify content, e.g., utilize a geographic location to modify the content selected for a given user such as a geographically relevant content summary (which may include doing an API call for a data summary based on a user's current location), a session-based content selection (e.g., to avoid repeatedly presenting nodes of a pathway already traversed), a language-based selection, etc.

In the example of FIG. 2A, a user is provided, e.g., via a browser hosting a web application, a display of part of pathway such as 102 of FIG. 1. Here it may be appreciated that the user has provided some contextual input data, for example answering an initial set of one or more questions regarding the progress made in forming and managing an IEP. As such, the user is directed by an embodiment, based on the data model 101 and its respective pathway 102, to a node 203, which with the user may choose to interact, e.g., select, hover over, touch, etc. As will be appreciated from FIG. 2A, the user is confined to move along the visually depicted pathway 202 such that access to additional content is minimized, i.e., in an embodiment a user may only select the nodes of a given pathway to display content. Thus, the user is confronted with a reduced set of navigable content selected based on the model and its designated pathway(s). As may be appreciated, a user may opt out of a given pathway, e.g., after being warned that the content pathway indicated has been chosen as most relevant based on the contextual data, etc.

As shown in FIG. 2B, a portion of the content associated with a respect element 203 of the interactive GUI 201 is chosen for display based on the user indication(s). In an embodiment, a user may navigate along one or more paths 202 defined by the respective data model, i.e., without a need to perform subsequent searches or refinements. That is, the interactive GUI 201 loads the one or more possible navigation paths 202 through the data for the user, guiding the user to specific parts of the larger data corpus. In this way, an embodiment facilitates efficient user interaction with a larger pool of data without requiring the user to know or understand relevant search terms, facets or available entry points into the data sets, etc.

Based on the information received from the user and document metadata values, e.g., metadata 104 referenced via input associated with element 203, an embodiment may dynamically retrieve or create a sub-set of the content from a specific data store, e.g., an IEP data store. In the example of FIG. 2B, an interactive user interface 204, such as a popup window or tool tip, new tab, window, etc., displays the dynamically created or retrieved content subset for the user via an interactive GUI 201. For example, the display might include an autogenerated or curated summary answering a displayed question text associated with the displayed data object 203 based on its relationship defined in the data model 101 with node 103.

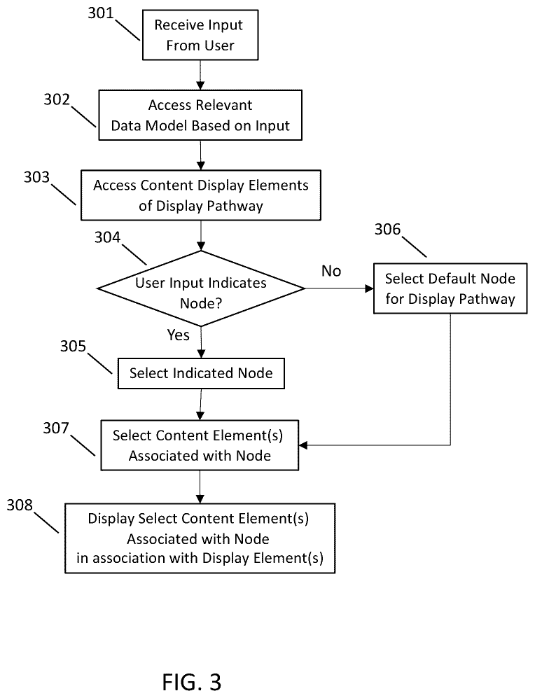

Shown in FIG. 3 is an example of a method provided by an embodiment. As illustrated, a system, such as an application server hosting a web application displayed via a browser of a client machine, may receive input from the user at 301. The input as described may be anonymous, e.g., a request for an initial webpage of a workflow dictating the paths of a data model, or may include contextual data, such as username, credentials, location data, proximity data, session data, etc.

Based on the received user input, an embodiment accesses the relevant data model based on the input at 302. For example, the data model may be preconfigured and associated with a static data element such as the page uniform resource locator (URL), such that it is accessed and used whenever the page associated with the static data element is called. Alternatively or additionally, the relevant data model may be accessed at 302 using additional data, e.g., the aforementioned contextual data, such as location, user profile information, or the like.

The data model accessed at 302 permits the identification of the related display elements, e.g., elements 202, 203 as illustrated in FIG. 2A, as shown at 303 such that the appropriate display objects may be loaded into the browser as interactable elements of interactive GUI 201. As indicated at 304, an embodiment may therefore determine whether the user input indicates a particular set of display data, such as a node, is indicated by the user input, as illustrated at 305. By way of example, an embodiment may determine that the user input is in response to a question posed by another interactable display element such as a question, the user's stored session data indicates a next-in-series node of a pathway, etc. If no such node is indicated, a default display set of data objects may be retrieved, for example a start page and its respective content, as indicated at 306.

In any case, an embodiment displays a set of data to the user in accordance with the respective data model and its pathways to govern the user's interaction with the data associated with the data model. Here it is indicated that an embodiment selects content element(s) at 307, e.g., content element 204, associated with the node(s) indicated, e.g., node 203, such that the interactive GUI 201 may display the selected content and display elements, which may or may not require user interaction with a display element, as indicated at 308.

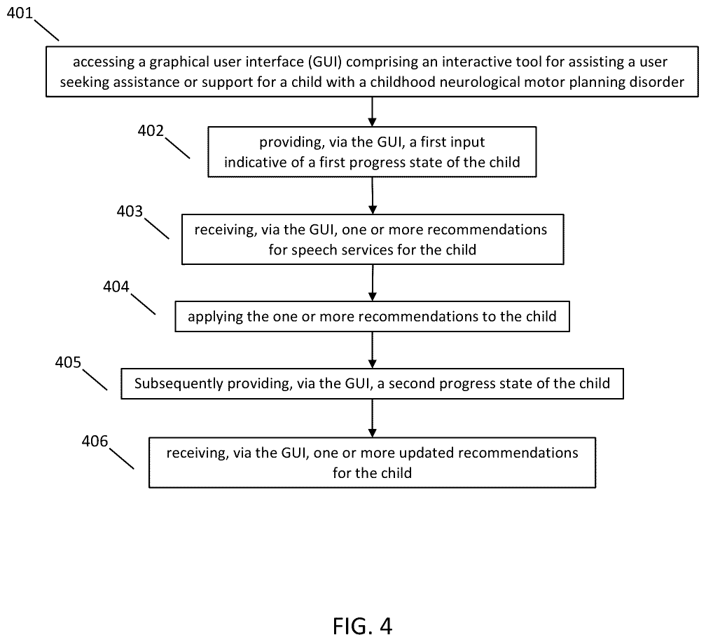

Referring to FIG. 4, a method is illustrated in which a GUI may be used as a tool by an end user, such as an IEP team member, for example a speech therapist, educator, parent, etc., to identify a current status or state of the child, e.g., progress towards forming or made in implementing an IEP and associated goals and services.

In an embodiment a method includes accessing a graphical user interface (GUI) comprising an interactive tool for assisting a user seeking assistance or support for a child with a childhood neurological motor planning disorder 401. A user provides, via the GUI, a first input indicative of a first progress state of the child 402. For example, a user may provide an input to a node posing a question relating to progress or adequacy of services currently being applied in an IEP or similar. The user input may for example indicate a lack of adequate progress or a belief that currently used and applied speech services are not optimal or should be revised. The user receives, via the GUI, one or more recommendations for speech services for the child 403. For example, the GUI may provide data in the form of recommending an adjustment to the IEP, recommend a speech service be utilized or requested, recommend at home speech practice, recommend further evaluation or referral, etc. The one or more recommendations are applied to the child 404. For example, a parent, guardian, speech language professional such as a therapist, etc., may use the tool to apply the speech service recommended to the child. Subsequently, the user provides, via the GUI, a second progress state of the child 405. For example, the user may access the GUI for a second time after a recommended speech service is utilized to report on progress or lack thereof and receive an updated recommendation. Thereafter, the user receives, via the GUI, one or more updated recommendations for the child 406.

In an embodiment, the childhood neurological motor planning disorder is childhood apraxia of speech and the one or more recommendations for speech services provided at 402 or updated recommendations at 406 comprise at home speech practice, for example to be implemented by an IEP team member such as a parent, training for school based staff, for example to be implemented for an IEP team member such as a teacher or therapist, or a combination of the foregoing. Further, recommendations for example received at 403 and/or 406 may include digital outputs that are readily converted into physical form, for example template letters, glossaries, etc. that may be printed on paper and used by the user.

In an embodiment, the method comprises providing, via the GUI, second input to one or more nodes representative of individual educational plan development stage, for example as outlined in model part 101 of FIG. 1. In an embodiment, the method comprises obtaining, via the GUI, content associated with a respective one of the one or more nodes associated with the second input, such as a recommendation. In an embodiment, the one or more nodes are logically associated along a guidance path such as 102 in FIG. 1. In an embodiment, the guidance path comprises a path for forming or updating an individual educational plan comprising the speech service, for example for receiving a recommendation for applying speech practice at home, for intensive speech services at school, or similar. In an embodiment, the method comprises receiving automated animation of displayed content along the guidance path in response to user input, as for example shown in FIG. 2A-B. In an embodiment, the method comprises obtaining a saved position in the guidance path for a user, e.g., by way of using context data at login or stored session information. Similarly, in an embodiment, the method comprises obtaining a customization of the guidance path based on one or more user characteristics of a user.

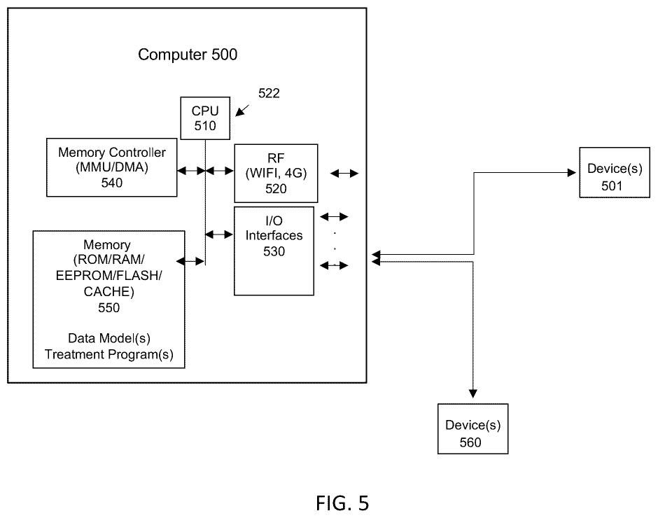

It will be readily understood that certain embodiments can be implemented using any of a wide variety of devices or combinations of devices. Referring to FIG. 5, an example device that may be used in implementing one or more embodiments includes a computing device (computer) 500.

The computer 500 may execute program instructions or code configured to store and process data (e.g., content, data models, and related display objects, as described herein) and perform other functionality of the embodiments. Components of computer 500 may include, but are not limited to, a processing unit 510, which may take a variety of forms such as a central processing unit (CPU), a graphics processing unit (GPU), a combination of the foregoing, etc., a system memory controller 540 and memory 550, and a system bus 522 that couples various system components including the system memory 550 to the processing unit 510. The computer 500 may include or have access to a variety of non-transitory computer readable media. The system memory 550 may include non-transitory computer readable storage media in the form of volatile and/or nonvolatile memory devices such as read only memory (ROM) and/or random-access memory (RAM). By way of example, and not limitation, system memory 550 may also include an operating system, application programs, other program modules, and program data. For example, system memory 550 may include application programs such as software including one or more data models and/or displayable pathways, as described herein. Data may be transmitted by wired or wireless communication, e.g., between computing devices, e.g., computer 500 and one or more remote devices or systems 501, 560, such as a cloud platform that provides web-based applications, 501, and data sources or interfaces for services 560, such as content summary services, or databases.

A user can interface with (for example, enter commands and information) the computer 500 through input devices such as a touch screen, keyboard, etc. A monitor or other type of display screen or device can also be connected to the system bus 522 via an interface, such as interface 530. The computer 400 may operate in a networked or distributed environment using logical connections to one or more other remote computers or databases. The logical connections may include a network, such local area network (LAN) or a wide area network (WAN) but may also include other networks/buses.

It should be noted that various functions described herein may be implemented using processor executable instructions stored on a non-transitory storage medium or device. A non-transitory storage device may be, for example, an electronic, electromagnetic, or semiconductor system, apparatus, or device, or any suitable combination of the foregoing. More specific examples of a non-transitory storage medium include the following: a portable computer diskette, a hard disk, a random-access memory (RAM), a read-only memory (ROM), an erasable programmable read-only memory (EPROM or Flash memory), a portable compact disc read-only memory (CD-ROM), a solid-state drive, or any suitable combination of the foregoing. In the context of this document “non-transitory” media includes all media except non-statutory signal media.

Program code embodied on a non-transitory storage medium may be transmitted using any appropriate medium, including but not limited to wireless, wireline, optical fiber cable, RF, etc., or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), a personal area network (PAN) or the connection may be made through other devices (for example, through the Internet using an Internet Service Provider), through wireless connections, or through a hard wire connection, such as over a USB or another power and data connection.

Example embodiments are described herein with reference to the figures, which illustrate various example embodiments. It will be understood that the actions and functionality may be implemented at least in part by program instructions. These program instructions may be provided to a processor of a device to produce a special purpose machine, such that the instructions, which execute via a processor of the device implement the functions/acts specified.

It is worth noting that while specific elements are used in the figures, and a particular illustration of elements has been set forth, these are non-limiting examples. In certain contexts, two or more elements may be combined, an element may be split into two or more elements, or certain elements may be re-ordered, re-organized, combined or omitted as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method, comprising:
accessing a graphical user interface (GUI) comprising an interactive tool for assisting a user seeking assistance or support for a child with a childhood neurological motor planning disorder;
providing, via the GUI, a first input indicative of a first progress state of the child;
receiving, via the GUI, one or more recommendations for speech services for the child;
applying the one or more recommendations to the child;
subsequently providing, via the GUI, a second progress state of the child; and
thereafter receiving, via the GUI, one or more updated recommendations for the child.

2. The method of claim 1, wherein the childhood neurological motor planning disorder is childhood apraxia of speech.

3. The method of claim 1, wherein the one or more recommendations for speech services comprise one or more of at home speech practice and intensive therapy services.

4. The method of claim 1, comprising providing, via the GUI, second input to one or more nodes representative of individual educational plan development stage.

5. The method of claim 4, comprising obtaining, via the GUI, content associated with a respective one of the one or more nodes associated with the second input.

6. The method of claim 5, wherein the one or more nodes are logically associated along a guidance path.

7. The method of claim 6, wherein the guidance path comprises a path for forming or updating an individual educational plan comprising the speech services.

8. The method of claim 6, comprising receiving automated animation of displayed content along the guidance path in response to user input.

9. The method of claim 6, comprising obtaining a saved position in the guidance path for a user.

10. The method of claim 9, comprising obtaining a customization of the guidance path based on one or more user characteristics of a user.
